SEQUENCE LISTING (9 sequences)

SEQ ID NO: 1 - protein, 248 aa, Unknown
  Description of Unknown Mannose-binding lectin (MBL) protein
MSLFPSLPLLLLSMVAASYSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 2 - protein, 228 aa, Unknown
  Description of Unknown Mannose-binding lectin (MBL) protein
ETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 3 - protein, 141 aa, Unknown
  Description of Unknown Mannose-binding lectin (MBL) protein
AASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 4 - protein, 115 aa, Unknown
  Description of Unknown Mannose-binding lectin (MBL) protein
VGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 5 - protein, 148 aa, Unknown
  Description of Unknown Mannose-binding lectin (MBL) protein
PDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 6 - protein, 380 aa, Artificial Sequence
  Description of Artificial Sequence Synthetic polypeptide
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 7 - protein, 383 aa, Artificial Sequence
  Description of Artificial Sequence Synthetic polypeptide
AKTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 8 - protein, 351 aa, Artificial Sequence
  Description of Artificial Sequence Synthetic polypeptide
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGATSKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 9 - protein, 232 aa, Artificial Sequence
  Description of Artificial Sequence Synthetic polypeptide
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGA

(19) United States
(12) Patent Application Publication
(10) Pub. No.: US 2016/0129053 A1
(43) Pub. Date: May 12, 2016

(54) METHODS OF ISOLATING MICROORGANISMS AND USES THEREOF

(71) Applicant: PRESIDENTS AND FELLOWS OF HARVARD COLLEGE, Cambridge, MA (US)

(72) Inventors: Laura BRASS, Newton, MA (US); Michael SUPER, Lexington, MA (US); Donald E. INGBER, Boston, MA (US)

(73) Assignee: PRESIDENT AND FELLOWS OF HARVARD COLLEGE, Cambridge, MA (US)

(21) Appl. No.: 14/893,675

(22) PCT Filed: May 23, 2014
(86) PCT No.: PCT/US2014/039277

§ 371 Date: Nov. 24, 2015

Related U.S. Application Data
(60) Provisional application No. 61/827,214, filed on May 24, 2013.

(51) Int. Cl. A61K 35/74; A61K 35/744; G01N 33/569; A61K 9/00

(52) U.S. Cl. CPC A61K 35/74; A61K 9/0053; A61K 35/744; G01N 33/56911; G01N 2333/33

(57) ABSTRACT

Provided herein are methods for preparing a microbiome sample for transplantation into a subject in need thereof. In particular, the methods and compositions relate to methods of repopulating the the microbiome of a subject in the treatment of gastrointestinal maladies e.g., diarrhea and/or constipation.

### CROSS-REFERENCE TO RELATED APPLICATIONS

This application claims benefit under 35 U.S.C. §119(e) of U.S. Provisional Application No. 61/827,214 filed May 24, 2013, the contents of which are incorporated herein by reference in their entirety.

### GOVERNMENT SUPPORT

This invention was made with government support under N66001-11-1-4180 awarded by United States Department of Defense/DARPA. The government has certain rights in the invention.

### TECHNICAL FIELD

The invention(s) described herein relate to supplementation and/or repopulation of the intestinal microbiome.

### BACKGROUND

Clostridium difficile (C. difficile) is the most frequent etiologic agent for health-care-associated diarrhea. It is estimated there are 3 million cases of Clostridium difficile infection (CDI) in the US each year, with 14,000 deaths.

CDI is usually associated with antibiotic treatment. C. difficile overgrows in the intestines and produces toxins that attack the intestinal lining, causing C. difficile colitis. C. difficile infection can range from mild to life-threatening. Symptoms of mild cases include watery diarrhea, three or more times a day for several days, with abdominal pain or tenderness. Symptoms of more severe C. difficile infection include watery diarrhea, up to 15 times each day, severe abdominal pain, loss of appetite, fever, bloody stool, and weight loss. In some cases, C. difficile infection can lead to a hole in the intestines, which can be fatal if not treated immediately.

The first-line treatment for patients is to stop administration of the antibiotics, and in more severe cases, treatment with a different antibiotic such as vancomycin is required. Up to 30% of patients have a recurrence of the infection, with subsequent recurrences at even higher rates.

### SUMMARY

The methods and compositions described herein are based, in part, on the discovery of a technique suitable for removing contaminants from a stool sample, thereby isolating a population of bacteria that can be used for repopulating the intestinal microbiome in a subject. Repopulation of the microbiome results in the treatment of a variety of gastrointestinal disorders, including diarrhea and/or constipation, in a subject.

In one aspect, the methods described herein relate to a method for isolating a plurality of bacteria from a biological sample, the method comprising: (a) contacting the sample with a plurality of opsonin or lectin molecules, wherein the plurality of opsonin or lectin molecules bind the plurality of bacteria in the sample, and (b) washing unbound material from the plurality of opsonin or lectin molecules, thereby isolating a plurality of bacteria from the sample.

In one embodiment of this aspect and all other aspects described herein, the opsonin or lectin molecule is a mannose-binding lectin molecule.

In one aspect, the methods described herein relate to a method for isolating a plurality of bacteria from a biological sample, the method comprising: (a) contacting the sample with a plurality of mannose-binding lectin (MBL) molecules, wherein the plurality of MBL molecules bind the plurality of bacteria in the sample, and (b) washing unbound material from the plurality of MBL molecules, thereby isolating a plurality of bacteria from the sample.

In one embodiment of this aspect and all other aspects described herein, the method further comprises a step of releasing the plurality of bacteria from the plurality of MBL molecules.

In another embodiment of this aspect and all other aspects described herein, the biological sample is a stool sample.

In another embodiment of this aspect and all other aspects described herein, the stool sample is obtained from a donor.

In another embodiment of this aspect and all other aspects described herein, the plurality of bacteria are representative of the bacteria present in the donor's gut.

In another embodiment of this aspect and all other aspects described herein, the donor is screened for pathogenic microbes.

In another embodiment of this aspect and all other aspects described herein, the plurality of MBL molecules are bound to a substrate.

In another embodiment of this aspect and all other aspects described herein, the method further comprises a step of selecting a donor and/or obtaining a sample from a selected donor.

In another embodiment of this aspect and all other aspects described herein, the isolated plurality of bacteria comprises viable bacteria.

In another embodiment of this aspect and all other aspects described herein, the plurality of bacteria are released from the plurality of MBL molecules by contacting with a calcium chelating agent.

In another embodiment of this aspect and all other aspects described herein, the biological sample or the isolated plurality of bacteria are tested for pathogens and/or allergens.

Also provided herein in another aspect is a method for treating a subject, the method comprising: administering the isolated plurality of bacteria, or portion thereof, made by a method as described herein, thereby treating the subject.

In one embodiment of this aspect, the method of isolating a plurality of bacteria comprises: (a) contacting the sample with a plurality of mannose-binding lectin (MBL) molecules, wherein the plurality of MBL molecules bind the plurality of bacteria in the sample, and (b) washing unbound material from the plurality of MBL molecules.

In another embodiment of this aspect and all other aspects described herein, the isolated plurality of bacteria, or portion thereof, further comprises a pharmaceutically acceptable carrier.

In another embodiment of this aspect and all other aspects described herein, the isolated plurality of bacteria are administered to the subject's intestines. In one embodiment of the methods and compositions described herein, the isolated plurality of bacteria are administered to a desired region of the intestines including, but not limited to the, ileum, proximal colon etc.

In another embodiment of this aspect and all other aspects described herein, the isolated plurality of bacteria are administered directly to the subject's intestines.

In another embodiment of this aspect and all other aspects described herein, the direct administration comprises a colonoscopy, nasogastric infusion, nasoduodenal infusion, or an enema.

In another embodiment of this aspect and all other aspects described herein, the isolated plurality of bacteria are administered using an enteric coated drug delivery device.

In another embodiment of this aspect and all other aspects described herein, the enteric coated drug delivery device comprises a capsule.

In another embodiment of this aspect and all other aspects described herein, the method further comprises removing or killing the existing bacteria in the subject's intestines prior to administering the isolated plurality of bacteria.

In another embodiment of this aspect and all other aspects described herein, the treatment is for diarrhea or constipation. In other embodiments, the treatment is for a disease or disorder selected from the group consisting of: inflammatory bowel disease, diarrhea, constipation (acute or chronic), irritable bowel syndrome, traveler's diarrhea, pediatric diarrhea, Crohn's disease, colitis, depression, anxiety, reduced immune function, obesity, irregularity, antibiotic-associated diarrhea, C. difficile infection, C. difficile relapse, lactose intolerance, colon cancer, hypercholesterolemia, hypertension, Helicobacter pylori infection, inflammation, skin infections, necrotizing fasciitis, Group B Strep. infection, Staph. aureus infection, yeast infection, opportunistic infections, yeast overgrowth, stress-induced bacterial growth, necrotizing enterocolitis, eczema, hyperglycemia, diabetes, insulin resistance, hypoglycemia, acne, psoriasis, failure to thrive, anemia, steatorrhea, abdominal distention with cramps, cramping, bloating, gas, dandruff, canker sores, rosacea, skin rashes, vaginitis, allergic contact dermatitis, ulcers, acid reflux, spastic colon, urinary tract infections, joint stiffness, and impaired vitamin production. See e.g., Goldin, BR and SL Gorbach (2008) 46 (S2): S96-S100, the contents of which are incorporated herein by reference in their entirety.

In another embodiment of this aspect and all other aspects described herein, the opportunistic infections are selected from a group consisting of: Acinetobacter baumanni, Aspergillus sp., Candida albicans, Clostridium difficile, Cryptococcus neoformans, Cryptosporidium, Cytomegalovirus, Geomyces destructans, Histoplasma capsulatum, Isospora belli, Legionnaires' Disease (Legionella pneumophila), Microsporidium, Mycobacterium avium complex (MAC) (Nontuberculosis Mycobacterium), Pneumocystis jirovecii, Pseudomonas aeruginosa, Staphylococcus aureus, Streptococcus pneumonia, Streptococcus pyogenes, and Toxoplasma gondii.

In another embodiment of this aspect and all other aspects described herein, the diarrhea is caused by pathogenic bacteria.

In another embodiment of this aspect and all other aspects described herein, the pathogenic bacteria comprises Clostridium difficile.

In another embodiment of this aspect and all other aspects described herein, the pathogenic bacteria are resistant to at least one antibiotic.

In another embodiment of this aspect and all other aspects described herein, the diarrhea caused by Clostridium difficile is a relapse of an initial C. difficile infection.

In another embodiment of this aspect and all other aspects described herein, the diarrhea and/or constipation is associated with irritable bowel syndrome, Crohn's disease, or colitis.

Another aspect provided herein relates to a composition comprising the isolated plurality of bacteria, or portion thereof, made by a method comprising the steps: (a) contacting the sample with a plurality of opsonin or lectin molecules, wherein the plurality of opsonin or lectin molecules bind the plurality of bacteria in the sample, and (b) washing unbound material from the plurality of opsonin or lectin molecules, thereby isolating a plurality of bacteria from the sample.

Another aspect provided herein relates to a composition comprising the isolated plurality of bacteria, or portion thereof, made by a method comprising the steps: (a) contacting the sample with a plurality of mannose-binding lectin (MBL) molecules, wherein the plurality of MBL molecules bind the plurality of bacteria in the sample, and (b) washing unbound material from the plurality of MBL molecules, thereby isolating a plurality of bacteria from the sample.

Also provided herein in another aspect is a pharmaceutical composition comprising a therapeutically effective amount of the isolated plurality of bacteria made by the method of the invention and a pharmaceutically acceptable carrier.

Also provided herein are uses of the isolated plurality of bacteria and pharmaceutical compositions thereof for the treatment of a subject having e.g., a gastrointestinal disease or disorder.

In one embodiment, the use of an isolated plurality of bacteria is contemplated for the treatment of a gastrointestinal disease or disorder, wherein the isolated plurality of bacteria are isolated by a method comprising: (a) contacting a biological sample with a plurality of mannose- binding lectin (MBL) molecules, wherein the plurality of MBL molecules bind the plurality of bacteria in the sample, and (b) washing unbound material from the plurality of MBL molecules, thereby isolating a plurality of bacteria from the sample.

### BRIEF DESCRIPTION OF THE DRAWINGS

FIG. 1 shows data from spike and recovery studies: spiking cecal contents (aerobic and anaerobic bacteria) into human blood and recovery with FcMBL beads. Various types of pathogens, including fungi (Ca; C.albicans), gram positive (Sa; S.aureus), negative (Ec; E.coli), and cecal inoculum. (Ci(an); cecal inoculum anaerobic, Ci(a);cecal inoculum aerobic), were spiked into human blood (10 mL) with 5 mM of CaCl₂ and then flowed through the blood cleansing device at a flow rate of 10 mL/h with FcMBL magnetic opsonins. Most of pathogens were removed from blood with efficiency above 90%, and about 80% of aerobic cecal inoculum in a single round of the blood cleansing process.

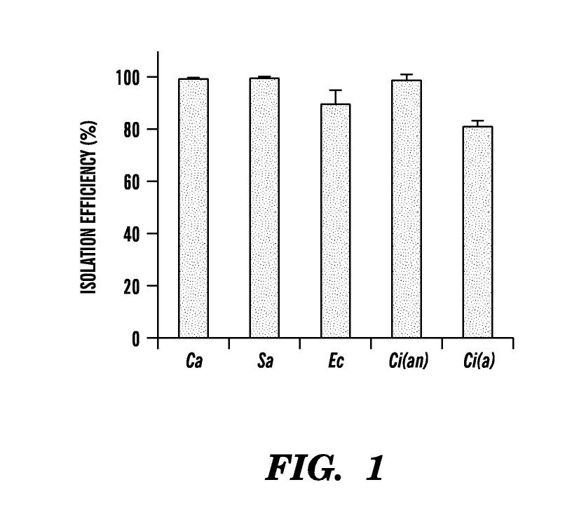

FIG. 2 shows data that indicate binding of FcMBL beads to cecal material in rat bloodstream in rat sepsis model. The FcMBL-Sandwich Pathogen ELISA can be used to detect a wide range of pathogens and can be used to diagnose sepsis in the rat model of intra-abdominal sepsis. (a) Diagnosis of Sepsis in rat model using FcMBL Pathogen ELISA experiment #1: Rat 1 was moribund and sacrificed at 11 hours, Rat #2 was moribund and sacrificed at 19 hours, the remaining rats survived to the end of the experiment at day 3. (b) Diagnosis of Sepsis in the Rat model of Intra-abdominal sepsis using the FcMBL Pathogen ELISA, Experiment #2: Rats # 1&2 were both moribund at 11 hours and were sacrificed, the remaining rats survived to the end of the experiment at day 5.

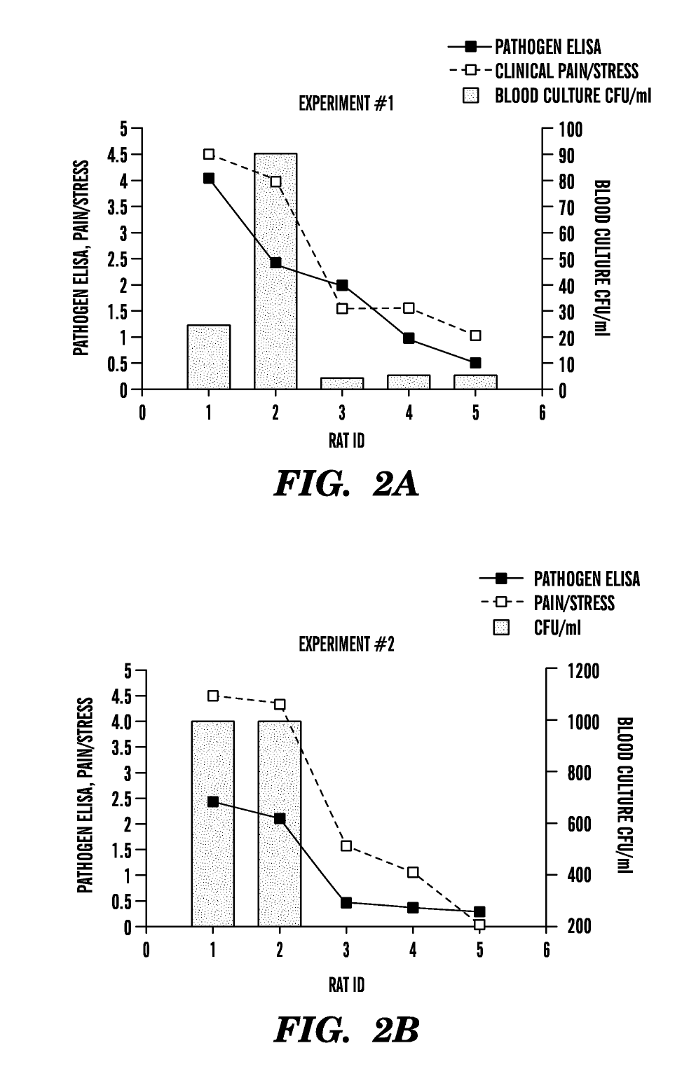

FIG. 3 shows data relating to binding of FcMBL to a wide range of bacteria, including common gut flora (E. coli, Enterococcus faecalis, Enterococcus facium, Enterobacter aerogenes, Enterobacter cloacae as well as Clostridium difficile. (Not to B. fragilis) 22 freshly isolated clinical strains of bacteria and fungi were tested using the FcMBL ELISA companion diagnostic test. 21 of the 22 strains were bound by the FcMBL beads and detected with the FcMBL HRP colorimetric reagent and only B. fragilis was not detected. In some cases these results differed from prior data collected with different FcMBL beads.

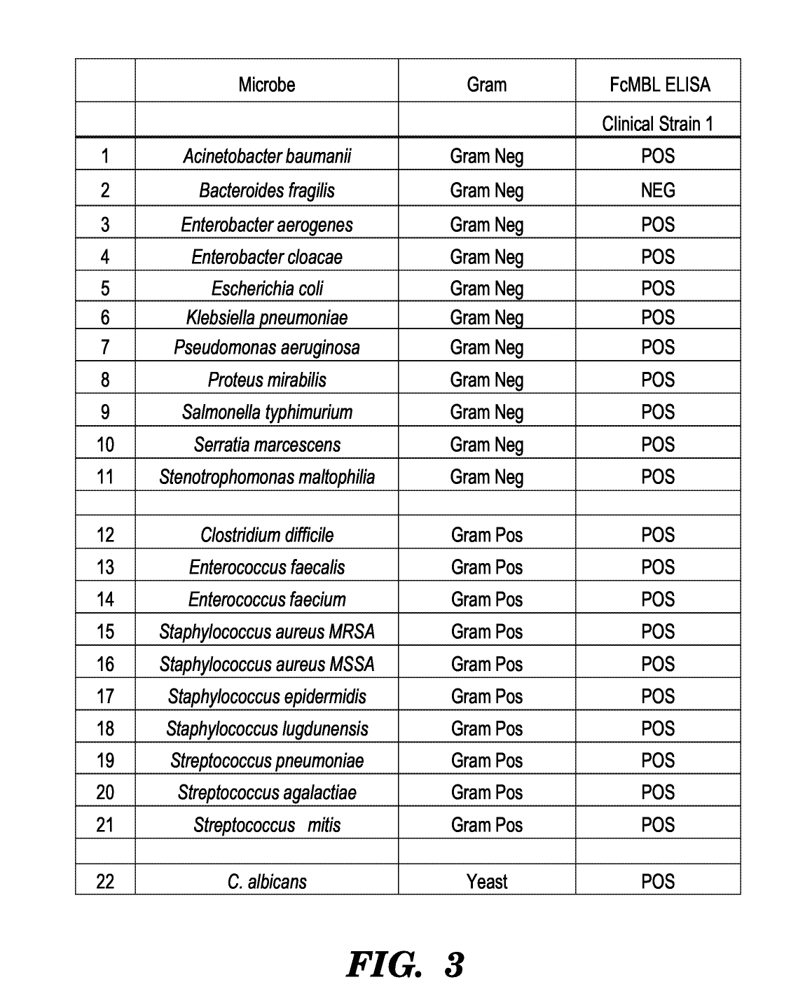

### DETAILED DESCRIPTION OF THE INVENTION

Provided herein are methods for preparing a microbiome sample for transplantation into a subject in need thereof. In particular, the methods and compositions relate to methods of repopulating the microbiome of a subject in the treatment of gastrointestinal maladies e.g., diarrhea and/or constipation. While the disclosure specifically uses mannose-binding lectin as a specific example, it is contemplated herein that any opsonin or lectin molecule can be used with the methods as described herein.

### DEFINITIONS

As used herein, a “subject” means a human or animal Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein.

In some embodiments, the subject is a mammal The mammal can be a human, non-human primate, mouse, rat, dog, cat, horse, or cow, but are not limited to these examples. Mammals other than humans can be advantageously used as subjects that represent animal models of disorders.

A subject can be one who has been previously diagnosed with or identified as suffering from or having a disease or disorder caused by any microbes or pathogens described herein. By way of example only, a subject can be diagnosed with C. difficile infection, Crohn's disease, inflammatory bowel disease, irritable bowel syndrome, or colitis.

As used herein, the term “donor” can refer to spouses or partners, first-degree relatives, other relatives, friends, or even individuals unknown to the subject to be treated. In one embodiment, the donor is a relative or family member of the subject to be treated. In another embodiment, the donor resides in the same household to the subject to be treated but is not necessarily a blood relation of the subject. When the subject is an animal, a donor refers to a blood relative of the animal or an animal housed in the same conditions as the subject, for example, treatment of a horse can be achieved using a donor that is housed in the same barn etc. or a donor that is related to the horse such as a parent or offspring of the horse. It is preferred that the donor is substantially free of pathogenic bacteria or other contaminants. By “substantially free” is meant below the level of acceptable guidelines for food contaminants as outlined by e.g., the FDA or Department of Agriculture. In one embodiment, “substantially free” refers to a level of the pathogenic bacteria or contaminants that is below detectable limits using standard techniques.

As used herein, the term “plurality” refers to at least two molecules (e.g., two MBL molecules) or at least two bacterial cells (e.g., the same or different species), for example, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, at least 100, at least 125, at least 150, at least 175, at least 200, at least 300, at least 400, at least 500, at least 600, at least 700, at least 800, at least 900, at least 1000, at least 1500, at least 2000, at least 5000, at least 10⁴, at least 10⁵, at least 10⁶, at least 10⁷, at least 10⁸, at least 10⁹, at least 10¹⁰ or more MBL molecules or bacterial cells.

As used herein, the term “representative of the bacteria present in the donor's gut” refers to an isolated plurality of bacteria that are present in a sample in substantially the same proportions as those present in the donor's gut and/or stool. A healthy microbiome is not simply any mixture of gut bacteria but also involves the proportion of the various bacteria in the gut, which work together for optimal digestive health.

As used herein, the term “viable bacteria” refers to bacterial cells that are capable of basic cellular functions including, e.g., growth, metabolism, division, and/or procreation. In one embodiment, the term “viable bacteria” also includes cells in a dormant state that under appropriate conditions can perform basic cellular functions.

As used herein the term “a portion thereof,” particularly relating to the isolated plurality of bacteria, or a portion thereof, refers to a subset of the isolated plurality of bacteria. That is, the entire quantity of the isolated plurality of bacteria need not be administered at one time or to one patient. One of skill in the art can use simple laboratory tests to determine the number of cells in the isolated plurality of bacteria and can infer the number of cells that will be needed for successful treatment of the subject based on the severity of the gastrointestinal infection or illness. The remaining portion that is not deemed necessary for use in immediate treatment of a subject can be stored for future use by the donor themselves or for future use by the same or a different subject (e.g., frozen or lyophilized) or can be used for diagnostic or research testing.

As used herein, the term “enteric coated drug delivery device” refers to any drug delivery method that can be administered orally but is not degraded or activated until the device enters the intestines. Such methods can utilize a coating or encapsulation that is degraded using pH dependent means, permitting protection of the delivery device and the microbiome to be transplanted throughout the upper gastrointestinal tract until the device reaches the alkaline pH of the intestines. In one embodiment, the enteric coated drug delivery device comprises a capsule or a pill. Such drug delivery devices are known to those of skill in the art.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used with the methods described herein that will be effective in the treatment of a particular disorder or condition will depend on the nature of the disorder or condition, and can be determined by standard clinical techniques.

As used herein, “prevention” or “preventing,” when used in reference to a disease, disorder or symptoms thereof, refers to a reduction in the likelihood that an individual will develop a disease or disorder, e.g., a hemoglobinopathy. The likelihood of developing a disease or disorder is reduced, for example, when an individual having one or more risk factors for a disease or disorder either fails to develop the disorder or develops such disease or disorder at a later time or with less severity, statistically speaking, relative to a population having the same risk factors and not receiving treatment as described herein. The failure to develop symptoms of a disease, or the development of reduced (e.g., by at least 10% on a clinically accepted scale for that disease or disorder) or delayed (e.g., by days, weeks, months or years) symptoms is considered effective prevention.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean ±1%.

The singular terms “a,” “an,” and “the” include plural referents unless context clearly indicates otherwise. Similarly, the word “or” is intended to include “and” unless the context clearly indicates otherwise. Thus for example, references to “the method” includes one or more methods, and/or steps of the type described herein and/or which will become apparent to those persons skilled in the art upon reading this disclosure and so forth.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

### Treatment of Diseases or Disorders Relating to a Lack of a Healthy Microbiome

Essentially any gastrointestinal disorder or other disorder relating to a lack of a healthy microbiome in a subject can be treated using the methods and compositions described herein. In one embodiment, the disease or disorder to be treated comprises diarrhea. Alternatively, the disease or disorder to be treated comprises constipation.

Diarrhea in a subject to be treated can be caused by a pathogenic bacterial infection (e.g., C. difficile infection), or can be due to an underlying condition present in the subject (e.g., inflammatory bowel disease). The underlying condition need not be a gastrointestinal condition as other disorders have been successfully treated using probiotics and are contemplated for treatment using the methods and compositions described herein. Thus, essentially any disease or disorder that can be treated with probiotics are also contemplated herein for treatment with the methods and compositions as described herein.

Some non-limiting conditions contemplated for treatment include inflammatory bowel disease, diarrhea, constipation (acute or chronic), irritable bowel syndrome, traveler's diarrhea, pediatric diarrhea, Crohn's disease, colitis, depression, anxiety, reduced immune function, obesity, irregularity, antibiotic-associated diarrhea, C. difficile infection, C. difficile relapse, lactose intolerance, colon cancer, hypercholesterolemia, hypertension, Helicobacter pylori infection, inflammation, skin infections, necrotizing fasciitis, Group B Strep. infection, Staph. aureus infection, yeast infection, opportunistic infections, yeast overgrowth, stress-induced bacterial growth, necrotizing enterocolitis, eczema, hyperglycemia, diabetes, insulin resistance, hypoglycemia, acne, psoriasis, failure to thrive, anemia, steatorrhea, abdominal distention with cramps, cramping, bloating, gas, dandruff, canker sores, rosacea, skin rashes, vaginitis, allergic contact dermatitis, ulcers, acid reflux, spastic colon, urinary tract infections, joint stiffness, and impaired vitamin production, among others.

In one embodiment, the condition to be treated using the methods and compositions described herein is obesity. For the treatment of overweight or obese subjects, a healthy or specific/desirable microbiota can be obtained from a selected donor, for example, a donor having a healthy weight (e.g., skinny, slim, athletic, fast metabolism etc.). For such an embodiment, the microbiome need not be optimal; that is a non-optimal but healthy microbiome would suffice for the methods and compositions described herein. Exemplary techniques for applying the methods and compositions as described herein for the treatment of obesity can be found in e.g., Park, DY. et al. PLOS One (2013) 8(3):e59470, Yook, SR et al. Obesity (Silver Spring) (2013) doi: 10.1002/oby.20428, Boroni Moreira, AP et al. Nutr Hosp (2012) 27(5):1408-1414, Safavi, M et al. Int J Food Sci Nutr (2013) Mar 12 epub ahead of print, Shen, J. et al. Mol Aspects Med (2013) 34(1):39-58, and Million, Met al. Curr Infect Dis Rep (2013) 15(1):25-30; the contents of each of the foregoing references are incorporated herein by reference in their entirety.

In some embodiments, the subjects to be treated are animals. It is contemplated herein that the methods and compositions described herein can be applied to animal having gastric issues, for example, ruminants with gastric issues. The methods and compositions described herein can be used to repopulate the gut in an animal following antibiotic administration or for treatment of an illness. Such methods can also be extended to permit animals to eat specific diets or certain foods that are not normally tolerated. Another example includes domestic pets or race horses. It is contemplated that the methods and compositions described herein can be used to treat or augment current treatments of race horses in preparation for race day Animals contemplated to be treated include e.g., cattle, horse, dogs, cats, pigs, sheep, goats, fowl, primates, bats, otters, seals, sea lions, penguins, elk, reindeer, deer, avians, reptiles, bears, moose, rodents, mice, rats, squirrels, chipmunks, ferrets, and the like.

The methods and compositions described herein can be used to augment the subject's existing gut microflora (e.g., by supplementation). Alternatively, the methods and compositions can be administered following a treatment to remove (e.g., intestinal lavage, enema, high dose or repeated rounds of laxatives etc.) or kill the existing microflora (e.g., treatment with one or more antibiotics). In one embodiment, one or more laxatives selected from the group consisting of: GOLYTELY, COLYTE, NULYTELY, phospho-soda, sodium phosphate tablets, OSMO-PREP and VISICOL are used for the elimination of fecal matter from the colon prior to transplantation using the methods as described herein. It is contemplated herein that removing or killing the existing bacteria in a subject is not a required step in transplantation of a microbiome to a subject.

### Selecting a Donor

One of skill in the art can select an appropriate donor for the methods and treatments as described herein. Typically, a donor is selected from the same geographical region as the subject to be treated, particularly because different geographical diets or conditions can have an impact on the microbiome of the donor. Thus, transplanting isolated bacteria from a donor in the same geographical region as the subject will increase the odds of a successful outcome in the subject. In addition, one might consider the dietary preferences of the subject when choosing a donor. For example, the microflora of a meat-eating individual is likely different from that of a vegetarian. Similarly, bacteria from a lactose intolerant donor may be insufficient for treatment of a subject who requires dairy in their diet. It may be preferable to select a donor that consumes a similar diet as the subject is expected to consume.

The donor can be a spouse or partner, a first-degree relative, another relative, a friend, or even an individual unknown to the subject to be treated. In one embodiment, the donor is a relative or family member of the subject to be treated. In another embodiment, the donor resides in the same household to the subject to be treated but is not necessarily a blood relation of the subject. In another embodiment, the compositions described herein can be prepared from a pool of donors.

### Obtaining a Sample

Any method known in the art can be used to obtain a sample from a suitable donor, provided that the sample is obtained in a manner that does not eliminate viability of at least a portion of the microbiome. Generally, the sample will be obtained by collecting stool of the suitable donor or other non-invasive means, however samples can be obtained from other areas of the body, including e.g., the skin. Donors should be instructed on proper collection and handling of the sample to preserve viability and/or prevent contamination of the sample with environmental contaminants, including pathogenic bacteria. In one embodiment, the samples are obtained in a hospital, clinic or other suitable facility.

The sample can be refrigerated or frozen prior to processing using the methods described herein, however it is generally preferred to process the sample as quickly as possible following collection to ensure maximum viability of the extracted microbiome.

Methods for fecal bacteriotherapy are routine, thus these and other considerations required for fecal bacteriotherapy are known to those of skill in the art. Other methods for fecal bacteriotherapy or treatment of intestinal maladies can be found in e.g., Brandt et al., Am J Gastroenterology (2012) 107:1079-1087 or Talaie, F et al. European Journal of Pharmaceutical Sciences (2013) entitled “Overcoming therapeutic obstacles in inflammatory bowel diseases: a comprehensive review on novel drug delivery strategies” in press, the contents of each are incorporated herein by reference in their entirety.

It is also contemplated herein that a microbiome can be obtained from a biological sample from any populated site of the donor's body including, but not limited to, intestines, skin, mouth and oral cavity, nasal cavity, ears, rectum, vagina, foreskin, or any mucus membrane.

### Opsonins

Provided herein are methods of isolating a plurality of cells (e.g., a microbiome) from a donor by contacting a biological sample obtained from the donor with at least one opsonin or lectin molecule. Essentially any opsonin or lectin can be used with the methods described herein. Such opsonins and lectins are known to those of skill in the art and can be selected based on the desired binding properties of the opsonin or lectin. In some embodiments, the methods described herein can use a heterogeneous mixture of at least two opsonins and/or lectins in a proportion suitable for isolating the microbiome as desired by one of skill in the art.

The term “opsonin” as used herein refers to naturally-occurring and synthetic molecules which are capable of binding to or attaching to the surface of a microbe or a pathogen, of acting as binding enhancers for a process of phagocytosis. Examples of opsonins which can be used in the engineered molecules described herein include, but are not limited to, vitronectin, fibronectin, complement components such as Clq (including any of its component polypeptide chains A, B and C), complement fragments such as C3d, C3b and C4b, mannose-binding protein, conglutinin, surfactant proteins A and D, C-reactive protein (CRP), alpha2-macroglobulin, and immunoglobulins, for example, the Fc portion of an immunoglobulin.

The term “lectin” as used herein refers to any molecules including proteins, natural or genetically modified (e.g., recombinant), that interact specifically with saccharides (e.g., carbohydrates). The term “lectin” as used herein can also refer to lectins derived from any species, including, but not limited to, plants, animals, insects and microorganisms, having a desired carbohydrate binding specificity. Examples of plant lectins include, but are not limited to, the Leguminosae lectin family, such as ConA, soybean agglutinin, peanut lectin, lentil lectin, and Galanthus nivalis agglutinin (GNA) from the Galanthus (snowdrop) plant. Other examples of plant lectins are the Gramineae and Solanaceae families of lectins. Examples of animal lectins include, but are not limited to, any known lectin of the major groups S-type lectins, C-type lectins, P-type lectins, and I-type lectins, and galectins. In some embodiments, the carbohydrate recognition domain can be derived from a C-type lectin, or a fragment thereof. C-type lectin can include any carbohydrate-binding protein that requires calcium for binding. In some embodiments, the C-type lectin can include, but are not limited to, collectin, DC-SIGN, and fragments thereof. Without wishing to be bound by theory, DC-SIGN can generally bind various microbes by recognizing high-mannose-containing glycoproteins on their envelopes and/or function as a receptor for several viruses such as HIV and Hepatitis C.

Collectins are soluble pattern recognition receptors (PRRs) belonging to the superfamily of collagen containing C-type lectins. Exemplary collectins include, without limitations, mannose-binding lectin (MBL) (also known as mannan-binding lectin, mannan-binding protein, or mannose-binding protein), surfactant protein A (SP-A), surfactant protein D (SP-D), collectin liver 1 (CL-L1), collectin placenta 1 (CL-P1), conglutinin, collectin of 43 kDa (CL-43), collectin of 46 kDa (CL-46), and a fragment thereof.

### Mannose-Binding Lectin Molecules

Mannose-binding lectin (MBL), also known as mannose binding protein (MBP), or mannan-binding lectin or mannan-binding protein, is a calcium-dependent serum protein that can play a role in the innate immune response by binding to carbohydrates on the surface of a wide range of microbes or pathogens (viruses, bacteria, fungi, protozoa) where it can activate the complement system. MBL can also serve as a direct opsonin and mediate binding and uptake of pathogens by tagging the surface of a pathogen to facilitate recognition and ingestion by phagocytes.

As used herein, the term “mannose-binding lectin molecule” or “MBL molecule” can refer to a carbohydrate recognition domain of an MBL, or a genetically engineered version of MBL (FcMBL) as described in International Application No. WO 2011/090954, filed Jan. 19, 2011, the contents of which are incorporated herein by reference in their entirety. Amino acid sequences for MBL and engineered MBL include, but are not limited to:

(i)
MBL full length (SEQ ID NO. 1):
MSLFPSLPLL LLSMVAASYS ETVTCEDAQK TCPAVIACSS
PGINGFPGKD GRDGTKGEKG EPGQGLRGLQ GPPGKLGPPG
NPGPSGSPGP KGQKGDPGKS PDGDSSLAAS ERKALQTEMA
RIKKWLTFSL GKQVGNKFFL TNGEIMTFEK VKALCVKFQA
SVATPRNAAE NGAIQNLIKE EAFLGITDEK TEGQFVDLTG
NRLTYTNWNE GEPNNAGSDE DCVLLLKNGQ WNDVPCSTSH
LAVCEFPI
(ii)
MBL without the signal sequence (SEQ ID NO. 2):
ETVTCEDAQK TCPAVIACSS PGINGFPGKD GRDGTKGEKG
EPGQGLRGLQ GPPGKLGPPG NPGPSGSPGP KGQKGDPGKS
PDGDSSLAAS ERKALQTEMA RIKKWLTFSL GKQVGNKFFL
TNGEIMTFEK VKALCVKFQA SVATPRNAAE NGAIQNLIKE
EAFLGITDEK TEGQFVDLTG NRLTYTNWNE GEPNNAGSDE
DCVLLLKNGQ WNDVPCSTSH LAVCEFPI
(iii)
Truncated MBL (SEQ ID NO. 3):
AASERKALQT EMARIKKWLT FSLGKQVGNK FFLTNGEIMT
FEKVKALCVK FQASVATPRN AAENGAIQNL IKEEAFLGIT
DEKTEGQFVD LTGNRLTYTN WNEGEPNNAG SDEDCVLLLK
NGQWNDVPCS TSHLAVCEFP I
(iv)
Carbohydrate recognition domain (CRD) of MBL
(SEQ ID NO. 4):
VGNKFFLTNG EIMTFEKVKA LCVKFQASVA TPRNAAENGA
IQNLIKEEAF LGITDEKTEG QFVDLTGNRL TYTNWNEGEP
NNAGSDEDCV LLLKNGQWND VPCSTSHLAV CEFPI
(v)
Neck + Carbohydrate recognition domain of MBL
(SEQ ID NO. 5):
PDGDSSLAAS ERKALQTEMA RIKKWLTFSL GKQVGNKFFL
TNGEIMTFEK VKALCVKFQA SVATPRNAAE NGAIQNLIKE
EAFLGITDEK TEGQFVDLTG NRLTYTNWNE GEPNNAGSDE
DCVLLLKNGQ WNDVPCSTSH LAVCEFPI
(vi)
FcMBL.81 (SEQ ID NO. 6):
EPKSSDKTHT CPPCPAPELL GGPSVFLFPP KPKDTLMISR
TPEVTCVVVD VSHEDPEVKF NWYVDGVEVH NAKTKPREEQ
YNSTYRVVSV LTVLHQDWLN GKEYKCKVSN KALPAPIEKT
ISKAKGQPRE PQVYTLPPSR DELTKNQVSL TCLVKGFYPS
DIAVEWESNG QPENNYKTTP PVLDSDGSFF LYSKLTVDKS
RWQQGNVFSC SVMHEALHNH YTQKSLSLSP GAPDGDSSLA
ASERKALQTE MARIKKWLTF SLGKQVGNKF FLTNGEIMTF
EKVKALCVKF QASVATPRNA AENGAIQNLI KEEAFLGITD
EKTEGQFVDL TGNRLTYTNW NEGEPNNAGS DEDCVLLLKN
GQWNDVPCST SHLAVCEFPI
(vii)
AKT-FcMBL (SEQ ID NO. 7):
AKTEPKSSDKTHT CPPCPAPELL GGPSVFLFPP
KPKDTLMISR TPEVTCVVVD VSHEDPEVKF NWYVDGVEVH
NAKTKPREEQ YNSTYRVVSV LTVLHQDWLN GKEYKCKVSN
KALPAPIEKT ISKAKGQPRE PQVYTLPPSR DELTKNQVSL
TCLVKGFYPS DIAVEWESNG QPENNYKTTP PVLDSDGSFF
LYSKLTVDKS RWQQGNVFSC SVMHEALHNH YTQKSLSLSP
GAPDGDSSLA ASERKALQTE MARIKKWLTF SLGKQVGNKF
FLTNGEIMTF EKVKALCVKF QASVATPRNA AENGAIQNLI
KEEAFLGITD EKTEGQFVDL TGNRLTYTNW NEGEPNNAGS
DEDCVLLLKN GQWNDVPCST SHLAVCEFPI
(viii)
FcMBL.111 (SEQ ID NO. 8):
EPKSSDKTHT CPPCPAPELL GGPSVFLFPP KPKDTLMISR
TPEVTCVVVD VSHEDPEVKF NWYVDGVEVH NAKTKPREEQ
YNSTYRVVSV LTVLHQDWLN GKEYKCKVSN KALPAPIEKT
ISKAKGQPRE PQVYTLPPSR DELTKNQVSL TCLVKGFYPS
DIAVEWESNG QPENNYKTTP PVLDSDGSFF LYSKLTVDKS
RWQQGNVFSC SVMHEALHNH YTQKSLSLSP GATSKQVGNKF
FLTNGEIMTF EKVKALCVKF QASVATPRNA AENGAIQNLI
KEEAFLGITD EKTEGQFVDL TGNRLTYTNW NEGEPNNAGS
DEDCVLLLKN GQWNDVPCST SHLAVCEFPI

MBL molecules can be attached to a solid substrate (e.g., beads) for use with the methods and compositions described herein. In one embodiment, the MBL molecules are attached to a solid substrate (e.g., beads) in the form of a purification column. Methods for using MBL molecules with the methods described herein can be found in e.g., International Patent Application Nos. WO/2011/090954 and WO/2013/012924, and U.S. Provisional Application No. 61/788,570, the contents of each are incorporated herein by reference in their entirety.

### Releasing Microbiota from MBL

Essentially any method of releasing cells from an MBL molecule as described in e.g., International Patent Application Nos. WO/2011/090954 and WO/2013/012924, and U.S. Provisional Application No. 61/788,570 (the contents of which are incorporated herein by reference in their entirety) can be used with methods and compositions described herein, provided that the method of releasing the cells from the MBL molecule permits the isolation of viable cells. In one embodiment, the method of releasing cells from the MBL molecule is a calcium dependent release mechanism using e.g., a calcium chelating agent as described in e.g., International Patent Application Nos. WO/2011/090954 and WO/2013/012924, and U.S. Provisional Application No. 61/788,570 (the contents of which are incorporated herein by reference in their entirety).

### Expansion and/or Culturing of Isolated Microbiome

It is also contemplated herein that the cells of the isolated microbiome as described herein are further expanded and/or cultured prior to preparation of a composition or transplantation to a subject in need thereof.

In some embodiments, the bacterial cells are cultured using a nutrient broth (a liquid nutrient medium) or LB medium (Lysogeny Broth) in the presence or absence of a solidifying agent such as agar. Other nutrients including amino acids, nitrogen, vitamins, co-factors, sugars, carbohydrates, minerals, salt, proteins, etc. can be added as necessary to the nutrient broths to improve the growth of the isolated microbiome. In one embodiment, such culturing methods can be used to optimize the amount of a particular bacterial species in the microbiome by selecting growth conditions for the desired bacterial strain.

One of skill in the art can expand and/or culture the isolated microbiome ex vivo or in vitro using standard laboratory methods or as described in e.g., Pathak, M. et al. Functional Foods in Health and Disease (2012) 2(10):369-378, which is incorporated by reference herein in its entirety. Other exemplary techniques are found in Heenan, CN. et al. Food Science and Technology (2002) 35(2):171-176, Chang, CP and SL Liew. Journal of Food Biochemistry (2012) doi: 10.1111/jfbc.1200, Corcoran, BM et al. Microbiology (2007) 153(1):291-299, International Patent Application WO2009/051509, U.S. Patent Application US2009/0087418, International Patent Application WO2002/012446, and International Patent Application WO2007/054989, the contents of each are incorporated by reference in their entirety.

### Additional Processing of Sample

It is also contemplated herein that the biological sample (e.g., stool sample) or the isolated plurality of bacteria can be processed further using standard laboratory techniques. For example, tests for common pathogenic microbes, viruses, immune cells, and contaminants can be performed on either the biological sample or the isolated bacteria. Such tests or assays are known to those of skill in the art.

In addition, the isolated plurality of bacteria can be further purified to remove unwanted contaminants prior to transplantation by using e.g., antibody mediated removal of specific contaminants including pathogenic microbes, opportunistic microbes, or immune cells from the donor.

### Contemplated Uses

The methods described herein are also contemplated for use as screening assays for potential donors. Alternatively, the methods described herein can be used in a screening assay to diagnose a disorder (e.g., gastrointestinal disorders) related to the lack of a healthy microbiome in the gut.

The methods described herein can also be used for research purposes for studying the microbiome in healthy patients or in subsets of patients suffering from a disease or disorder.

In addition, the methods described herein can be used to study the microbiome for the purpose of designing an optimal probiotic e.g., for commercialization. For example, one of skill in the art can analyze the number of cells necessary for a healthy microbiome in an individual as well as the proportion of gut bacteria for the purposes of designing a probiotic for dietary supplementation. It is contemplated that such supplements will be designed in e.g., a cultural, regional and/or seasonal manner. Such designer probiotics would permit maximum efficacy that would complement regional diets, seasonal considerations and even culturally associated dietary requirements. One of skill in the art can design such supplements for use in e.g., the general population. Also contemplated herein are probiotic supplements made by the methods described herein.

In one embodiment, the methods and compositions described herein can be used to treat and/or prevent a bad stomach while traveling (e.g., traveler's diarrhea). This can be achieved by transplanting the microbiome from a donor that lives in a region to which the subject will be or is traveling.

In another embodiment, the isolated microbiome as described herein is co-administered with a therapeutic concentration of antibiotic including, but not limited to: Gentamicin; Vancomycin; Oxacillin; Tetracyclines; Nitroflurantoin; Chloramphenicol; Clindamycin; Trimethoprim-sulfamethoxasole; a member of the Cephlosporin antibiotic family (e.g., Cefaclor, Cefadroxil, Cefixime, Cefprozil, Ceftriaxone, Cefuroxime, Cephalexin, Loracarbef, and the like); a member of the Penicillin family of antibiotics (e.g., Ampicillin, Amoxicillin/Clavulanate, Bacampicillin, Cloxicillin. Penicillin VK, and the like); with a member of the Fluoroquinolone family of antibiotics (e.g., Ciprofloxacin, Grepafloxacin, Levofloxacin, Lomefloxacin, Norfloxacin, Ofloxacin, Sparfloxacin, Trovafloxacin, and the like); or a member of the Macrolide antibiotic family (e.g., Azithromycin, Erythromycin, and the like).

Similarly, the isolated microbiome as described herein can be co-administered with a therapeutically-effective concentration of an anti-fungal. Such anti-fungal agents include, but are not limited to: Clotrimazole, Fluconazole, Itraconazole, Ketoconazole, Miconazole, Nystatin, Terbinafine, Terconazole, and Tioconazole. By co-administration is meant sequentially or simultaneously, in the same or different compositions.

### Pharmaceutical Compositions

The methods of administering an isolated microbiome to a subject as described herein involve the use of therapeutic compositions comprising a population of isolated bacteria from a donor. Therapeutic compositions contain a physiologically tolerable carrier together with the cell composition and optionally at least one additional bioactive agent as described herein, dissolved or dispersed therein as an active ingredient. In a preferred embodiment, the therapeutic composition is not substantially immunogenic when administered to a mammal or human patient for therapeutic purposes, unless so desired.

In one embodiment, the isolated plurality of cells described herein (e.g., microbiome, gut flora, or intestinal microflora) are administered as a suspension with a pharmaceutically acceptable carrier. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation comprising cells can include e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the isolated plurality of cells as described herein using routine experimentation.

A cell composition can also be emulsified or presented as a liposome composition, provided that the emulsification procedure does not adversely affect cell viability. The cells and any other active ingredient can be mixed with excipients which are pharmaceutically acceptable and compatible with the active ingredient and in amounts suitable for use in the therapeutic methods described herein.

Additional agents included in a cell composition as described herein can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions as described herein that is effective in the treatment of a particular disorder or condition will depend on the nature of the disorder or condition, and can be determined by standard clinical techniques.

### Dosage and Administration

In one aspect, the methods described herein provide a method for repopulating a microbiome in a subject. In one embodiment, the subject can be a mammal In another embodiment, the mammal can be a human, although the approach is effective with respect to all mammals. The method comprises administering to the subject an effective amount of a pharmaceutical composition comprising an isolated plurality of bacteria as described herein, in a pharmaceutically acceptable carrier. In one embodiment, the pharmaceutical composition comprises a fiber supplement (e.g., psyillium).

The dosage range for the transplanted bacteria depends upon the potency, and include amounts large enough to produce the desired effect, e.g., improvement in diarrhea or constipation in a treated subject. The dosage should not be so large as to cause unacceptable adverse side effects. Generally, the dosage will vary with the type of illness, and with the age, condition, and sex of the patient. The dosage can be determined by one of skill in the art and can also be adjusted by the individual physician in the event of any complication. Typically, the dosage ranges from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain serum levels between 5 μg/mL and 30 μg/mL.

For use in the various aspects described herein, an effective amount of an isolated plurality of cells as described herein, comprises at least 10² bacterial cells, at least 1×10³ bacterial cells, at least 1×10⁴ bacterial cells, at least 1×10⁵ bacterial cells, at least 1×10⁶ bacterial cells, at least 1×10⁷ bacterial cells, at least 1×10⁸ bacterial cells, at least 1×10⁹ bacterial cells, at least 1×10¹⁰ bacterial cells, at least 1×10¹¹ bacterial cells, at least 1×10¹² bacterial cells or more. The bacterial microbiome cells can be derived from one or more donors, or can be obtained from an autologous source. In some embodiments of the aspects described herein, the cells of the isolated microbiome are expanded in culture prior to administration to a subject in need thereof.

Administration of the doses recited above can be repeated for a limited period of time. In some embodiments, the doses are given once a day, or multiple times a day, for example but not limited to three times a day. In a preferred embodiment, the doses recited above are administered daily for several weeks or months. The duration of treatment depends upon the subject's clinical progress and responsiveness to therapy. Continuous, relatively low maintenance doses are contemplated after an initial higher therapeutic dose.

The term “effective amount” as used herein refers to the amount of a population of isolated cells or their progeny needed to alleviate at least one or more symptom of a disease or disorder relating to a lack of a healthy microbiome (e.g., diarrhea), and relates to a sufficient amount of a composition to provide the desired effect, e.g., treat a subject having diarrhea. The term “therapeutically effective amount” therefore refers to an amount of bacterial cells or a composition comprising such cells that is sufficient to promote a particular effect when administered to a typical subject, such as one who has or is at risk for a gastrointestinal disorder or ailment. An effective amount as used herein would also include an amount sufficient to prevent or delay the development of a symptom of the disease, alter the course of a symptom disease (for example but not limited to, slow the progression of a symptom of the disease), or reverse a symptom of the disease. It is understood that for any given case, an appropriate “effective amount” can be determined by one of ordinary skill in the art using routine experimentation.

A therapeutically effective amount is an amount of an agent that is sufficient to produce a statistically significant, measurable change in a symptom relating to lack of a healthy microbiome. Such effective amounts can be gauged in clinical trials as well as animal studies for a given agent.

Agents useful in the methods and compositions described herein can be administered topically, intravenously (by bolus or continuous infusion), orally, by inhalation, intraperitoneally, intramuscularly, subcutaneously, intracavity, and can be delivered by peristaltic means, if desired, or by other means known by those skilled in the art.

Therapeutic compositions containing at least one agent can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology. In some embodiments, an agent can be targeted to a tissue by using bispecific antibodies, for example produced by chemical linkage of an anti-ligand antibody (Ab) and an Ab directed toward a specific target. To avoid the limitations of chemical conjugates, molecular conjugates of antibodies can be used for production of recombinant bispecific single-chain Abs directing ligands and/or chimeric inhibitors at cell surface molecules. The addition of an antibody to an agent permits the agent to accumulate additively at the desired target site (e.g., intestines or region thereof). Antibody-based or non-antibody-based targeting moieties can be employed to deliver a ligand or the inhibitor to a target site. Preferably, a natural binding agent for an unregulated or disease associated antigen is used for this purpose.

Precise amounts of active ingredient required to be administered depend on the judgment of the practitioner and are particular to each individual. However, suitable dosage ranges for are disclosed herein and depend on the route of administration.

In the preparation of pharmaceutical formulations comprising an isolated microbiome as disclosed herein in the form of dosage units for oral administration the compound selected can be mixed with solid, powdered ingredients, such as lactose, saccharose, sorbitol, mannitol, starch, arnylopectin, cellulose derivatives, gelatin, fiber (e.g., psyillium) or another suitable ingredient, as well as with disintegrating agents and lubricating agents such as magnesium stearate, calcium stearate, sodium stearyl fumarate and polyethylene glycol waxes. The mixture is then processed into granules or pressed into tablets.

Soft gelatin capsules can be prepared with capsules containing a mixture of the isolated cells in vegetable oil, fat, or other suitable vehicle for soft gelatin capsules. Hard gelatin capsules can also contain the cells in combination with solid powdered ingredients such as lactose, saccharose, sorbitol, mannitol, potato starch, corn starch, arnylopectin, cellulose derivatives or gelatin.

Dosage units for rectal or vaginal administration can be prepared (i) in the form of suppositories which contain the isolated microbiome mixed with a neutral fat base; (ii) in the form of a gelatin rectal capsule which contains the cells in a mixture or suspension with a vegetable oil, paraffin oil or other suitable vehicle for gelatin rectal capsules; (iii) in the form of a ready-made micro enema; or (iv) in the form of a dry micro enema formulation to be reconstituted in a suitable solution just prior to administration.

Liquid preparations for oral administration can be prepared in the form of syrups or suspensions, e.g., solutions or suspensions containing from 0.2% to 20% by weight of cells and the remainder consisting of e.g., sugar or sugar alcohols and a mixture of ethanol, water, glycerol, propylene glycol and polyethylene glycol. If desired, such liquid preparations can contain coloring agents, flavoring agents, saccharin and carboxymethyl cellulose or other thickening agents. Liquid preparations for oral administration can also be prepared in the form of a dry powder to be reconstituted with a suitable solution prior to use.

Solutions for parenteral administration can be prepared as a solution of a compound of the invention in a pharmaceutically acceptable solvent, preferably in a concentration from 0.1% to 10% by weight. These solutions can also contain stabilizing ingredients and/or buffering ingredients and are dispensed into unit doses in the form of ampoules or vials. Solutions for parenteral administration can also be prepared as a dry preparation to be reconstituted with a suitable solvent extemporaneously before use.

As used herein, the terms “administering,” “introducing” and “transplanting” are used interchangeably in the context of the placement of cells, e.g. an isolated microbiome, as described herein into a subject, by a method or route which results in at least partial localization of the introduced cells at a desired site, such as the intestines or a region thereof, such that a desired effect(s) is produced. The cells, or their differentiated progeny can be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years, i.e., long-term engraftment.

In some embodiments, the isolated microbiome as described herein is combined with one or more probiotic organisms prior to treatment of a subject. As used herein, the term “probiotic” refers to microorganisms that form at least a part of the transient or endogenous flora and thereby exhibit a beneficial prophylactic and/or therapeutic effect on the host organism. Probiotics are generally known to be clinically safe (i.e., nonpathogenic) by those individuals skilled in the art. By way of example, and not of limitation to any particular mechanism, the prophylactic and/or therapeutic effect of a lactic acid-producing bacteria and/or the microbiome as described herein is due, in part, from a competitive inhibition of the growth of pathogens due to: (i) their superior colonization abilities; (ii) parasitism of undesirable microorganisms; (iii) the production of lactic acid and/or other extracellular products possessing anti-microbial activity; or (iv) various combinations thereof. It should be noted that the aforementioned products and activities of the lactic acid-producing bacteria of the present invention act synergistically to produce the beneficial probiotic effect disclosed herein. Typical lactic acid-producing bacteria useful as a probiotic of this invention are efficient lactic acid producers which include non-pathogenic members of the Bacillus genus which produce bacteriocins or other compounds which inhibit the growth of pathogenic organisms. Exemplary lactic acid-producing, non-pathogenic Bacillus species include, but are not limited to: Bacillus coagulans; Bacillus coagulans Hammer; and Bacillus brevis subspecies coagulans.

Exemplary lactic acid-producing Lactobacillus species include, but are not limited to: Lactobacillus acidophilus, Lactobacillus casei, Lactobacillus DDS-1, Lactobacillus GG, Lactobacillus rhamnosus, Lactobacillus plantarum, Lactobacillus reuteri, Lactobacillus gasserii, Lactobacillus jensenii, Lactobacillus delbruekii, Lactobacillus, bulgaricus, Lactobacillus salivarius and Lactobacillus sporogenes (also designated as Bacillus coagulans).

Exemplary lactic acid-producing Sporolactobacillus species include all Sporolactobacillus species, for example, Sporolactobacillus P44.

Exemplary lactic acid-producing Bifidiobacterium species include, but are not limited to: Bifidiobacterium adolescentis, Bifidiobacterium animalis, Bifidiobacterium bifidum, Bifidiobacterium bifidus, Bifidiobacterium breve, Bifidiobacterium infantis, Bifidiobacterium infantus, Bifidiobacterium longum, and any genetic variants thereof.

Examples of suitable non-lactic acid-producing Bacillus include, but are not limited to: Bacillus subtilis, Bacillus uniflagellatus, Bacillus lateropsorus, Bacillus laterosporus BOD, Bacillus megaterium, Bacillus polymyxa, Bacillus licheniformis, Bacillus pumilus, and Bacillus sterothermophilus. Other strains that could be employed due to probiotic activity include members of the Streptococcus (Enterococcus) genus. For example, Enterococcus faecium, is commonly used as a livestock probiotic and, thus, could be utilized as a co-administration agent. Furthermore, it is also intended that any of the acid-producing species of probiotic or nutritional bacteria known in the art can be used in the compositions comprising an isolated microbiome as described herein.

A nutrient supplement comprising the isolated microbiome as described herein can include any of a variety of nutritional agents, including vitamins, minerals, essential and nonessential amino acids, carbohydrates, lipids, foodstuffs, dietary supplements, and the like. Preferred compositions comprise vitamins and/or minerals in any combination. Vitamins for use in a composition as described herein can include vitamins B, C, D, E, folic acid, K, niacin, and like vitamins. The composition can contain any or a variety of vitamins as may be deemed useful for a particularly application, and therefore, the vitamin content is not to be construed as limiting. Typical vitamins are those, for example, recommended for daily consumption and in the recommended daily amount (RDA), although precise amounts can vary. The composition can preferably include a complex of the RDA vitamins, minerals and trace minerals as well as those nutrients that have no established RDA, but have a beneficial role in healthy human or mammal physiology. The preferred mineral format would include those that are in either the gluconate or citrate form because these forms are more readily metabolized by lactic acid bacteria. In a related embodiment, the compositions described herein are contemplated to comprise an isolated microbiome in combination with a viable lactic acid bacteria in combination with any material to be adsorbed, including but not limited to nutrient supplements, foodstuffs, vitamins, minerals, medicines, therapeutic compositions, antibiotics, hormones, steroids, and the like compounds where it is desirable to insure efficient and healthy absorption of materials from the gastrointestinal track into the blood. The amount of material included in the composition can vary widely depending upon the material and the intended purpose for its absorption, such that the composition is not to be considered as limiting.

In some embodiments, the compositions described herein can further include a prebiotic and/or a fiber. As used herein, the term “prebiotic” includes substances or compounds that beneficially affect the host mammal by selectively promoting the growth and/or activity of one or more probiotic bacterial in the gastro-intestinal tract of the host mammal, thus maintaining normal health or improving health of the host. Typically, prebiotics are carbohydrates, (such as oligosaccharides), but the term “prebiotic” as used herein does not preclude non-carbohydrates. Many forms of “fiber” exhibit some level of prebiotic effect. Thus, there is considerable overlap between substances that can be classified as “prebiotics” and those that can be classified as “fibers”. Non-limiting examples of prebiotics suitable for use in the compositions and methods include psyllium, fructo-oligosaccharides, inulin, oligofructose, galacto-oligosaccharides, isomalto-oligosaccharides xylo-oligosaccharides, soy-oligosaccharides, gluco-oligosaccharides, mannan-oligosaccharides, arabinogalactan, arabinxylan, lacto sucrose, gluconannan, lactulose, polydextrose, oligodextran, gentioligosaccharide, pectic oligosaccharide, xanthan gum, gum arabic, hemicellulose, resistant starch and its derivatives, and mixtures and/or combinations thereof. The compositions can comprise from about 100 mg to about 100 g, alternatively from about 500 mg to about 50 g, and alternatively from about 1 g to about 40 g, of prebiotic, per day or on a less than daily schedule.

### Efficacy Measurement

The efficacy of a given treatment for a subject lacking a healthy microbiome can be determined by the skilled clinician. However, a treatment is considered “effective treatment,” as the term is used herein, if any one or all of the signs or symptoms of the e.g., gastrointestinal malady is/are altered in a beneficial manner, other clinically accepted symptoms or markers of disease are improved, or even ameliorated, e.g., by at least 10% following treatment with an agent that comprises an isolated microbiome as described herein. Efficacy can also be measured by a failure of an individual to worsen as assessed by stabilization of the e.g., gastrointestinal disease or disorder, hospitalization or need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of the disease; or (2) relieving the disease, e.g., causing regression of symptoms; and (3) preventing or reducing the likelihood of the development of the malady.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

One option for treatment of patients with recurrent and severe C. difficile infection (CDI) is a fecal microbiota or microbiome transplantation, where the microbiota (microbial community of the gut) from a healthy donor is transplanted to the colon of the CDI patient. Fresh stool samples from the donor are taken, processed, and delivered either orally or directly to the colon via a colonoscope. A recent study indicates that fecal microbiota transplantation has a 91% cure rate (Brandt et al., Am J Gastroenterology (2012) 107:1079-1087, the contents of which are incorporated herein by reference in their entirety).

Provided herein are methods for purifying a microbiota or gut flora from a donor, for example, to remove unwanted material, such as chemical or biological contaminants, pathogens, allergens or other immune stimulating factors prior to administration of the microbiota to the subject.

In one embodiment, the methods described herein can be used to take the microbiota out of healthy donor samples, purify the microbes and encapsulate the microbiota in pill/capsule form with an enteric coating that dissolves only in the colon. The compositions made by such a method are easier to store (e.g., frozen, lyophilized, etc.) and can avoid the need for a colonoscopy delivery method.

One advantage of using the methods described herein is that using a plurality of MBL molecules (e.g., an MBL column) can remove immune cells, factors that are immunogenic or allergenic, and other undesirable things from the microbiota prior to transplantation. With a purified microbiota sample, one can also perform tests on it to make sure it is not contaminated with pathogens or other unwanted material, and optionally one can also remove undesirable material (for example, remove any C. difficile that are be present in a sample for example by binding them to specific antibodies, etc.). Such methods can also permit the use of autologous fecal transplants by removing pathogenic bacteria from the patient's own stool before transplanting it back. Generally when a subject is suffering from a pathogenic infection, it is preferred to obtain the microbiota from a healthy donor since a healthy microbiota involves much more than simply removing pathogenic bacteria; rather it involves re-populating the gut with a healthy balance of beneficial bacteria, which is generally lacking in this population due to prior treatment with antibiotics or due to a compromised immune system.

Another advantage of the methods described herein is that the methods permit isolation of viable bacteria (e.g., live and intact), that can optionally be cultured prior to transplantation of the microbiota population. The MBL-captured microbiota can be given to the subject (e.g., a subject infected with C. difficile) orally, in a pill or capsule form, with the colonoscope, or any other method known to those of skill in the art.

The methods and compositions described herein should not be construed as being limited to treatment of C. difficile infection. Such methods and compositions can also be for treatment of inflammatory bowel disease, irritable bowel syndrome, Crohn's disease, and other maladies associated with an unhealthy gut microflora. It is also contemplated that microbiota from thin people can be administered to obese patients (as has been shown in some mouse studies). The methods compositions described herein can also be administered to patients before the onset of antibiotic treatments or major surgeries—in this case the microbiota could be from a donor or could be autologous.

Also contemplated herein are diagnostic tests for C. difficile using the methods described herein. Since MBL binds to C. Difficile, the methods described herein can also be used to remove C. difficile from samples and also as a diagnostic for the presence of C. difficile in samples or on hospital surfaces, etc. Using MBL to purify the microbiota can enable profiling and analysis of the populations of bacteria in the microbiota and the further study of the microbiome.

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

SEQUENCE LISTING:

MSLFPSLPLL LLSMVAASYS ETVTCEDAQK TCPAVIACSS PGINGFPGKD GRDGTKGEKG
EPGQGLRGLQ GPPGKLGPPG NPGPSGSPGP KGQKGDPGKS PDGDSSLAAS ERKALQTEMA
RIKKWLTFSL GKQVGNKFFL TNGEIMTFEK VKALCVKFQA SVATPRNAAE NGAIQNLIKE
EAFLGITDEK TEGQFVDLTG NRLTYTNWNE GEPNNAGSDE DCVLLLKNGQ WNDVPCSTSH
LAVCEFPI (SEQ ID NO. 1)
ETVTCEDAQK TCPAVIACSS PGINGFPGKD GRDGTKGEKG EPGQGLRGLQ GPPGKLGPPG
NPGPSGSPGP KGQKGDPGKS PDGDSSLAAS ERKALQTEMA RIKKWLTFSL GKQVGNKFFL
TNGEIMTFEK VKALCVKFQA SVATPRNAAE NGAIQNLIKE EAFLGITDEK TEGQFVDLTG
NRLTYTNWNE GEPNNAGSDE DCVLLLKNGQ WNDVPCSTSH LAVCEFPI (SEQ ID NO. 2)
AASERKALQT EMARIKKWLT FSLGKQVGNK FFLTNGEIMT FEKVKALCVK FQASVATPRN
AAENGAIQNL IKEEAFLGIT DEKTEGQFVD LTGNRLTYTN WNEGEPNNAG SDEDCVLLLK
NGQWNDVPCS TSHLAVCEFP I (SEQ ID NO. 3)
VGNKFFLTNG EIMTFEKVKA LCVKFQASVA TPRNAAENGA IQNLIKEEAF LGITDEKTEG
QFVDLTGNRL TYTNWNEGEP NNAGSDEDCV LLLKNGQWND VPCSTSHLAV CEFPI
(SEQ ID NO. 4)
PDGDSSLAAS ERKALQTEMA RIKKWLTFSL GKQVGNKFFL TNGEIMTFEK VKALCVKFQA
SVATPRNAAE NGAIQNLIKE EAFLGITDEK TEGQFVDLTG NRLTYTNWNE GEPNNAGSDE
DCVLLLKNGQ WNDVPCSTSH LAVCEFPI (SEQ ID NO. 5)
EPKSSDKTHT CPPCPAPELL GGPSVFLFPP KPKDTLMISR TPEVTCVVVD VSHEDPEVKF
NWYVDGVEVH NAKTKPREEQ YNSTYRVVSV LTVLHQDWLN GKEYKCKVSN KALPAPIEKT
ISKAKGQPRE PQVYTLPPSR DELTKNQVSL TCLVKGFYPS DIAVEWESNG QPENNYKTTP
PVLDSDGSFF LYSKLTVDKS RWQQGNVFSC SVMHEALHNH YTQKSLSLSP GAPDGDSSLA
ASERKALQTE MARIKKWLTF SLGKQVGNKF FLTNGEIMTF EKVKALCVKF QASVATPRNA
AENGAIQNLI KEEAFLGITD EKTEGQFVDL TGNRLTYTNW NEGEPNNAGS DEDCVLLLKN
GQWNDVPCST SHLAVCEFPI (SEQ ID NO. 6)
AKTEPKSSDKTHT CPPCPAPELL GGPSVFLFPP KPKDTLMISR TPEVTCVVVD VSHEDPEVKF
NWYVDGVEVH NAKTKPREEQ YNSTYRVVSV LTVLHQDWLN GKEYKCKVSN KALPAPIEKT
ISKAKGQPRE PQVYTLPPSR DELTKNQVSL TCLVKGFYPS DIAVEWESNG QPENNYKTTP
PVLDSDGSFF LYSKLTVDKS RWQQGNVFSC SVMHEALHNH YTQKSLSLSP GAPDGDSSLA
ASERKALQTE MARIKKWLTF SLGKQVGNKF FLTNGEIMTF EKVKALCVKF QASVATPRNA
AENGAIQNLI KEEAFLGITD EKTEGQFVDL TGNRLTYTNW NEGEPNNAGS DEDCVLLLKN
GQWNDVPCST SHLAVCEFPI (SEQ ID NO. 7)
EPKSSDKTHT CPPCPAPELL GGPSVFLFPP KPKDTLMISR TPEVTCVVVD VSHEDPEVKF
NWYVDGVEVH NAKTKPREEQ YNSTYRVVSV LTVLHQDWLN GKEYKCKVSN KALPAPIEKT
ISKAKGQPRE PQVYTLPPSR DELTKNQVSL TCLVKGFYPS DIAVEWESNG QPENNYKTTP
PVLDSDGSFF LYSKLTVDKS RWQQGNVFSC SVMHEALHNH YTQKSLSLSP GATSKQVGNKF
FLTNGEIMTF EKVKALCVKF QASVATPRNA AENGAIQNLI KEEAFLGITD EKTEGQFVDL
TGNRLTYTNW NEGEPNNAGS DEDCVLLLKN GQWNDVPCST SHLAVCEFPI (SEQ ID NO. 8)
EPKSSDKTHT CPPCPAPELL GGPSVFLFPP KPKDTLMISR TPEVTCVVVD VSHEDPEVKF
NWYVDGVEVH NAKTKPREEQ YNSTYRVVSV LTVLHQDWLN GKEYKCKVSN KALPAPIEKT
ISKAKGQPRE PQVYTLPPSR DELTKNQVSL TCLVKGFYPS DIAVEWESNG QPENNYKTTP
PVLDSDGSFF LYSKLTVDKS RWQQGNVFSC SVMHEALHNH YTQKSLSLSP GA
(SEQ ID NO. 9)

What is claimed is:

1. A method for isolating a plurality of bacteria from a biological sample, the method comprising:
(a) contacting the sample with a plurality of mannose-binding lectin (MBL) molecules, wherein the plurality of MBL molecules bind the plurality of bacteria in the sample, and
(b) washing unbound material from the plurality of MBL molecules, thereby isolating a plurality of bacteria from the sample.

2. The method of claim 1, further comprising a step of releasing the plurality of bacteria from the plurality of MBL molecules.

3. The method of claim 1, wherein the biological sample is a stool sample.

4-6. (canceled)

7. The method of claim 1, wherein the plurality of MBL molecules are bound to a substrate.

8. The method of claim 1, further comprising a step of selecting a donor and/or obtaining a sample from a selected donor.

9. The method of claim 1, wherein the isolated plurality of bacteria comprises viable bacteria.

10. The method of claim 1, wherein the plurality of bacteria are released from the plurality of MBL molecules by contacting with a calcium chelating agent.

11. The method of claim 1, wherein the biological sample or the isolated plurality of bacteria are tested for pathogens and/or allergens.

12. A method for treating a subject, the method comprising: administering a composition comprising the isolated plurality of bacteria of claim 1, or a portion thereof, thereby treating the subject.

13. The method of claim 12, wherein the composition further comprises a pharmaceutically acceptable carrier.

14. The method of claim 12, wherein the isolated plurality of bacteria are administered to the subject's intestines.

15-18. (canceled)

19. The method of claim 12, further comprising removing or killing the existing bacteria in the subject's intestines prior to administering the isolated plurality of bacteria.

20. The method of claim 12, wherein the treatment is for diarrhea or constipation.

21. The method of claim 20, wherein the diarrhea is caused by pathogenic bacteria.

22. The method of claim 21, wherein the pathogenic bacteria comprises Clostridium difficile.

23. The method of claim 21, wherein the pathogenic bacteria are resistant to at least one antibiotic.

24. The method of claim 20, wherein the diarrhea is caused by Clostridium difficile and is a relapse of an initial C. difficile infection.

25. The method of claim 20, wherein the diarrhea and/or constipation is associated with irritable bowel syndrome, Crohn's disease, or colitis.

26. A composition comprising the isolated plurality of bacteria of claim 1, or a portion thereof.

27. A pharmaceutical composition comprising a therapeutically effective amount of the isolated plurality of bacteria of claim 1 and a pharmaceutically acceptable carrier.

28-30. (canceled)
